(19) United States
(12) Patent Application Publication
(10) Pub. No.: US 2023/0346101 A1
(43) Pub. Date: Nov. 2, 2023

(54) TREATMENT OF FUNGAL NAIL INFECTION

(71) Applicant: Distinct Dermatology Inc., Parsippany, NJ (US)

(72) Inventors: Jeffrey Strauss, Whippany, NJ (US); Trung Pham, Royersford, PA (US)

(73) Assignee: Distinct Dermatology Inc., Parsippany, NJ (US)

(21) Appl. No.: 18/133,290

(22) Filed: Apr. 11, 2023

Related U.S. Application Data
(60) Provisional application No. 63/336,996, filed on Apr. 29, 2022.

(51) Int. Cl. A45D 29/17; A61K 31/137; A61K 31/145; A61K 31/4418; A61K 31/454; A61K 31/69

(52) U.S. Cl. CPC A45D 29/17; A61K 31/137; A61K 31/145; A61K 31/4418; A61K 31/454; A61K 31/69

(57) ABSTRACT

This invention relates to a treatment of fungal nail infections. The fungal infection is treated by debriding the nail to remove debris and fungus from the infected nail. A dispersion layer is applied on the debridement, and then a layer of UV adhesive layer is cured over the dispersion layer, wherein both layers may include effective amounts of anti-fungal medication. The treated nail may be further cosmetically treated.

### CROSS REFERENCE TO PRIOR APPLICATION

The present application claims the benefit of and priority, under 35 U.S.C. § 119(e), to U.S. Provisional Application Ser. No. 63/336,996, filed on Apr. 29, 2022, entitled “TREATMENT OF ANTIFUGAL INFECTION,” which is incorporated herein by reference in its entirety for all that it teaches and for all purposes.

### BACKGROUND

Fungal infections commonly affect skin and nails, and nail infections occur more commonly in toenails than fingernails. The most common nail fungal infection is onychomycosis. The majority of onychomycosis cases are caused by dermatophytes, e.g., Trichophyton Rubrum. Onychomycosis may also be caused by nondermatophyte molds e.g., Aspergillus, Scopulariopsis, and Fusarium. Onychomycosis occurs in about 10% of the general population with most of the infections occurring to those over the age of 70. Anyone can get a fungal nail infection, but those with a nail injury, weaken immune system, and poor circulation due to diabetes, may be more suspectable to a fungal nail infection. Typically, an infection occurs when a fungus enters between a nail and a nail bed though a cracked or damaged nail. Once the fungus enters between the nail and nail bed, the fungus feeds on keratin to grow.

Onychomycosis is a disease that is typically characterized by nail thickening, splitting of the nails, the nail separating from the nail bed, yellowish discoloration of the nails, brittleness and crumbling of the nails, deformation of the nail shape, detachment of the nails, embarrassing order, and pain in the fingers and toes. If the infected nails are not treated, secondary bacterial infection around the infected nails, such as fingers and toes, may occur. Onychomycosis is also transmissible between humans. Onychomycosis may not be a serious disease, but the infection may be painful and many of those infected are embarrassed by the physical side-effects of their nails such as disfigured nails and foul smell. The embarrassment prevents those infected with onychomycosis from enjoying outdoor activities, wearing sandals and open toe shoes, or simply refusing to go bare feet.

U.S. Pat. No. 8,409,558 discloses a method of treating onychomycosis by topically applying a layer of alkyl cyanoacrylate to an infected nail. The method applies a layer of alkyl cyanoacrylate to the surface of a debrided nail, but not under the nail plate, i.e., the nail bed. It is theorized that alkyl cyanoacrylate absorbs moisture and air in the nail, which are required for fungus growth. The patent also discloses that an anti-fungal agent may be added to the alkyl cyanoacrylate liquid composition.

However, the alkyl cyanoacrylate treatment has limitations. The debridement of the infected nail and an application of the alkyl cyanoacrylate require patients to revisit a medical professional or nail technician multiple times and sometime multiple times in a single week. The re-debridement and re-application of the alkyl cyanoacrylate are required as earliest as 3 days after the initial treatment. The alkyl cyanoacrylate treatment may last between 2 to 12 months. Requiring patients to receive multiple treatments a week and potentially up to a year is time consuming, inconvenient, and a financial burden.

In addition, although alkyl cyanoacrylate layer may remove (absorb) moisture from the infected nail to limit fungus growth, moisture may still be present under the nail, i.e., moisture from the skin or introduced after the nail is cut. Also, a medical professional or nail technician only has seconds to apply the alkyl cyanoacrylate, aka “instant glue,” before it cures upon exposure to air. It is difficult to properly apply an effective amount of alkyl cyanoacrylate layer on the infected nail in such a short amount of time without over/under or misapplying the alkyl cyanoacrylate to ensure that all moisture is removed from the infected nail. Accidental “gluing” such as gluing of fingers, tools or other objects is also a common occurrence.

### SUMMARY

The present document discloses a method of treating a fungal nail infection. The treatment creates a fungistatic environment on the fungal infected nail. The treatment method may include debriding a nail plate of a fungal infected nail to remove nail plate debris and an encapsulated fungus. The debridement step may be a chemical debridement with 40% urea. After debriding the step, an application of a dispersion layer impregnated with an anti-fungal agent may be applied on the debrided nail to inhibit the growth and spread of the fungal infection. A layer of UV adhesive may be applied on the dispersion layer to cover the dispersion layer and the debrided nail to seal the dispersion layer and to add depth and rigidity. The UV adhesive layer is cured with UV light. Further, the treatment may include cosmetically treating the fungal infected nail by trimming and shaping the dispersion layer and UV adhesive layer.

In another aspect, a method of treating a fungal nail infection may include debriding a nail plate of a fungal infected nail to remove nail plate debris and an encapsulated fungus. As part of or after the debridement step at least one of baking soda, an abrasive material, sodium percarbonate, and citric acid may be used to scrub the fungal infected nail. The debridement step may be a chemical debridement with 40% urea. The infected nail is further cleaned and disinfected with isopropyl alcohol. After the debridement step, a mesh dispersion layer impregnated with an anti-fungal agent may be applied on the debrided nail to inhibit the growth and spread of the fungal infection and to create a fungistatic environment. A layer of UV adhesive may be applied on the dispersion layer to cover the dispersion layer and the debrided nail to seal the dispersion layer and to add depth and rigidity. The UV adhesive layer is cured with UV light. Further, the treatment may include cosmetically treating the infected nail by trimming and shaping the dispersion layer and UV adhesive layer.

In another aspect, a method of treating a fungal nail infection may include debriding a nail plate of a fungal infected nail to remove nail plate debris and an encapsulated fungus. As part of or after the debridement step at least one of baking soda, an abrasive material, sodium percarbonate, and citric acid may be used to scrub the fungal infected nail. The debridement step may be a chemical debridement with 40% urea. The infected nail is further cleaned and disinfected with isopropyl alcohol. After the debridement step, an alcohol-based anti-fungal topical layer may be applied on the debrided nail to inhibit the growth and spread of the fungal infection to create a fungistatic environment. A layer of UV adhesive may be applied on the dispersion layer to cover the dispersion layer and the debrided nail to seal the dispersion layer and to add depth and rigidity. The UV adhesive layer is cured with UV light. Further, the treatment may include cosmetically treating the fungal infected nail by trimming and shaping the dispersion layer and UV adhesive layer.

The features described above and further detailed in description may be included in any combination as understood by a person of ordinary skilled in the art.

### DETAIL DESCRIPTION

In the following detailed descriptions, specific details are set forth to understand exemplary embodiments of this invention. It is understood that the certain examples and embodiments disclosed in the detailed description should not be used to limit the scope of the invention. It is further understood that terms used in the following detailed descriptions are used in their ordinary and accustomed meanings in the art, unless otherwise specified.

Fungal nail infections, such as onychomycosis is difficult to cure even with proper treatment. A treatment must be started early in the infection stages for best results. The treatment may last for months and sometimes up to a year or more. Even after proper treatment and cure, it is common for the fungal nail infection to return.

Oral and topical medications are available for the treatment of fungal nail infections. In general, oral medications are more effective compared to topical medications. The goal of fungal infection treatments is to remove the organisms that cause the infection. A treatment via oral medications is a long process that can take more than one year to cure, and the treatment can be expensive. Terbinafine and fluconazole are examples of well-known oral medications. Oral medications also have the risk of negative drug interactions. Beta blockers antiarrhythmic, antipsychotics, and antihistamines drugs are some of the known drugs that interacts with the oral medications. Oral medications require careful monitoring of liver enzymes due to the risk of liver damage.

Topical anti-fungal medications include creams, solutions, powder, gel, sprays, and lacquer containing effective amounts of antifungal agents such as ciclopirox, amorolfine, or other antifungal agents. Typically, topical medications are over the counter (OTC) medications and do not require a prescription. Accordingly, topical anti-fungal medications are used for mild fungal infections. Clinical studies have shown less than 30% cure rate among patients using only topical treatment for fungal nail infections. Accordingly, topical medications are less effective than oral medications. Another drawback of the topical medications is that once the topical medication is applied, the treated nail cannot be washed, nor can the nail come in contact with water for several hours.

Oral and topical medications are only moderately effective in removing organisms that cause fungal infections such as onychomycosis. In addition, the medications do not treat the thickening, splitting, discoloring, brittleness, deformation, and detachment of the nail caused by the fungal nail infection. In addition to the traditional fungal infection treatments, there are other therapies, such as laser, iontophoresis, UV light and photodynamic therapy. However, these therapies are expensive and there is no hard evidence that these treatments are substantially more effective compared to traditional medicines.

Treating a fungal infection of the nails requires more than simply treating the infection. Most available fungal nail infection treatments only target removing the fungal infection. These treatments do not alleviate the side-effects of the infections. Fungal infections may cause pain in the infected nails, aesthetically embarrassing split nails, yellowish discoloration of the nails, deformation of the nail shape, and foul order. Those with fungal nail infections and side-effects are prevented from enjoying common activities such as wearing open toe shoes, going bare foot on beaches and parks, and showing their bare feet to others. The present disclosure teaches a novel treatment that not only effectively treats fungal nail infections, but also effectively remedies the common side-effects of the fungal nail infections.

A fungal nail infection, such as onychomycosis, may be suspected due to the characterization of the infection, for example, nail thickening, splitting of the nails and the nails separating from the nail bed, yellowish discoloration of the nails, brittleness and crumbling of the nails, deformation of the nail shape, detachment of the nails, foul order, and pain in the toes. Not all fungal nail infections require treatment, and some treatments may be self-treated with topical OTC medications. Persistent and severe fungal nail infections may require a visit to and a treatment by a medical professional. Some infections may be treated by a primary care physician, but the more severe and complicated infections may require a specialist such as a dermatologist or podiatrist.

Onychomycosis may be suspected by a simple observation of the infected nail, but a proper diagnosis is critical in successfully identifying and treating onychomycosis or any fungal nail infections. A patient suspected of onychomycosis may seek medical attention for diagnosis and proper treatment. A potassium hydroxide (KOH) test may be conducted on the suspected infected nail. The suspected nail is disinfected, clipped, and observed under microscope for fungal infections. Additional diagnostic techniques may be used, such as culturing, polymerase chain reaction (PCR), and histopathologic examination of periodic acid-Schiff (PAS)—stained nail clippings and subungual debris.

A patient suspected of having a nail infection may also seek diagnosis and treatment from an on-line telehealth service website. The on-line telehealth service, such as Getmystride.com, is a HIPPA compliant on-line medical consultation service and treatment provider that specializes in fungal nail infections. A patient seeking a telehealth treatment first visits an on-line website. Patients with moderate infections may self-treat by purchasing footcare products such as topical applications, foot creams, foot masks, supplements, etc., from the on-line store. Patients with persistent and/or severe fungal nail infections may sign-up for the “Toenail Fungus Treatment” on the on-line telehealth service provider's website and seek medical consultation from the website's professional healthcare provider. Patients that seek the on-line telehealth services complete a consultation form, i.e., an intake form. The intake form including photos of the affected area are reviewed by the professional healthcare provider to confirm that the patient has a fungal infection and to determine the severity of the infection. Based on the review and conclusion of the intake form, the patient is prescribed a treatment regimen.

A client of a nail salon may be advised by a professional nail technician that the client is suspected of having a fungal nail infection. The client seeks medical attention for diagnosis and proper treatment either through a traditional medical process, i.e., visiting a medical profession at their office, or the client may visit an on-line telehealth service provider such as Getmystride.com. After it is clinically confirmed by a medical professional that the client (patient) is infected with a fungal nail infection such as onychomycosis, the patient may receive a treatment regimen from a professional nail technician at the nail salon based on strict guidelines and instructions provided by the medical professional. The professional nail technician provides no medical diagnosis, makes no medical determinations, nor provides any medical advice. The processional nail technician only follows strict guidelines and instructions provided by the medical professional. The nail technician's expertise and experience in the practice of nail technology, i.e., practice of manicuring nails, is highly suitable in the treatment process. The guidelines and instructions provided by the medical professional may include a fungal infection nail treatment kit. The treatment kit may include disposable or non-disposal nail debridement tools. The treatment kit may further include necessary disinfectant and cleaning solutions, dispersion layer ingredients/materials having active pharmaceutical ingredients (API) therein, and an UV adhesive with an UV LED flashlight, and other necessary supplies. The treatment kit includes all the necessary supplies for a specific treatment regimen tailored to the patient based on the clinical determination and diagnosis. Again, the nail technician only follows strict guidelines and instructions provided by the medical professional in the proper use and application of the treatment kit. In addition to a professional nail technician, a medical professional may also use the treatment kit.

After a clinical determination that a patient has a fungal nail infection such as onychomycosis, a treatment regimen to treat and inhibit the growth and spread of the fungal infection may commence. A treatment may start with a nail debridement to remove encapsulated fungus and diseased portions including loose debris on the infected nail. As part of the nail debridement step, the infected nail may be soaked in warm water. Afterwards, dead skin cells near the infected nail and cuticles may be removed. Trimming the end of the nails by cutting or grounding may also be required. Nail debridement removes and reduces the thickness and length of the infected nail. The debridement should be performed by a trained professional such as a podiatrist or a nail technician. The infected nail may be manually debrided with tools such as nail cutters, clippers, nail files, curettes, push sticks, cuticle trimmers, etc. The infected nail may also be debrided using electric tools such as a Dremel and a sander. In addition to or as an alternative to a mechanical debridement, a chemical debridement may be performed on the infected nail with greater than 25% urea, in particular between 30%-40% urea. Studies have shown that 40% urea is effective in dissolving infected nails but not healthy nails. The urea softens and hydrates the nail plate, which assists in enhancing the passage of anti-fungal agent to the infected area by denaturing the nail keratin and thus enhancing the drug penetration and promoting the avulsion of the infected nails. Chemical debridement also has the advantage of having less side effects such as secondary bacterial infections caused by mechanical debriding tools via accidental cuts to the skin near the infected nail. The chemical debridement process is relatively painless, which is ideal for those with arthritis or nerve damage on the toes and fingers due to diabetes.

The nail debridement step is performed to remove loose nail debris around the affected area. Depending on the level of fungal infection, the debridement step may remove loose nail debris, remove part of the nail plate (dorsal layer) around the infected area, or remove the entire nail plate. The debridement step may expose the nail bed. In addition to removing the diseased portion, the debridement step provides a stable substrate for subsequent treatment steps, e.g., a dispersion layer and an adhesive layer.

The debridement step may further include scrubbing/brushing the infected nail with baking soda or similar abrasive material. Baking soda or other abrasive materials help loosen and exfoliate the nail. Baking soda also have anti-fungal properties. The baking soda may be scrubbed/brushed with a brush. In addition to the baking soda scrubbing/brushing, the nail may be further treated with citric acid, e.g., diluted lemon concentrate. Citric acid has also been shown to have anti-fungal properties and may further prevent the spread of the fungal infection. Citric acid is also a non-irritant to the skin near the nail, which may be advantageous to those with sensitive skin.

After the debridement step or as part of the debridement step, the infected nail may be further cleaned with 70% isopropyl alcohol for disinfection to prevent secondary infections. Sodium percarbonate as a bleaching agent may also be used to treat nails having substantial discoloration. Sodium percarbonate also has cleaning and anti-fungal properties.

After the debridement and cleaning of the infected nail, a dispersion layer is applied to the debrided nail. The dispersion layer may be a transdermal drug delivery system (TDDS) layer. A TDDS is a route of delivering active pharmaceutical ingredients (API) to a specific area, such as an infected nail, over an extended time. The dispersion layer prevents the spread of the fungal infection by creating a fungistatic environment.

A porous material such as a mesh having an effective amount of API may be used as the dispersion layer. Fibrous materials such as polymeric nanofibers have shown excellent drug loading capabilities. Polymeric nanofibers have high surface area are porous and nanosized for easy incorporation. An example of a polymeric nanofibers is a nanofiber mesh composed of poly(e-caprolactone) (PCL), which is a biodegradable aliphatic polyester. The mesh dispersion layer also creates a strong bond with an adhesive layer.

A transdermal patch may also be used as the dispersion layer. Transdermal patches adhere to the skin, i.e., debrided nail, to deliver API to the infected area over an extended time. Transdermal patches provide a non-invasive and painless drug delivery system. Transdermal patches typically have an adhesive backing which is used to adhere and secure to the applied skin. In exemplary embodiments of the present disclosure, an adhesive backing on the transdermal patches may not be required. The API of the transdermal patch is formulated to permeate onto the infected area and/or into the blood stream. The length of time and dosage of the API delivery vary depending on need. The API delivery may also be enhanced with chemical enhancers and permeators for faster delivery. Transdermal patches deliver effective amounts of API over an extend amount of time, thereby removing the need for periodic reapplications.

Microsomes, liposomes, and fiberglass with API may also be used as the dispersion layer. Microsponages have a cross-linked organic framework and have been used to control the release of API. Microsponages are porous, elastic, and have capillary action for excellent drug delivery capacities. Liposomes are nanometric-sized delivery systems composed of phospholipids, which are key components of cell membranes, making liposomes well tolerated and devoid of toxicity on the skin and nails. Fiberglass may also be used as the dispersion layer. A topical application of alcohol with an antifungal API, e.g., terbinafine, may also create a fungistatic environment, thus can also be used as the dispersion layer.

A dispersion layer, for example, a thin layer of mesh, a transdermal patch, sponge, liposomes, a topical application or fiberglass is formulated to deliver an effective amount of anti-fungal agent such as OTC strength or prescription strength terbinafine 1%, tolnaftate 1% or prescription strength ciclopirox 8%, efinaconazole 10%, and tavaborole 5%. The dispersion layer also serves to aesthetically cover the infected nail by adding depth to the debrided nail. If excess amount of the nail plate is debrided or if there is substantial chipping of the infected nail, a layer of natural cellulose fiber may be applied to or under the dispersion layer for added depth and surface area.

An application of an adhesive layer is applied over the dispersion layer. The adhesive layer may be a layer of UV light sensitive epoxy resin or acrylate (UV adhesive). The UV adhesive is applied and then cured by a UV light to seal the dispersion layer and the debrided nail. The UV adhesive layer seals the dispersion layer, further adds depth, and rigidity to the debrided nail.

Unlike traditional strong fast acting adhesives such as alkyl cyanoacrylate, aka “instant glue,” which cures immediately upon exposure to air, UV adhesives do not cure upon exposure to air but only cures when exposed to UV light. The UV wavelength creates a photochemical reaction in the UV adhesives to cure the UV adhesives. The benefits of the UV adhesives include a consistent application and a less likelihood of accidental and unintended “gluing” of objects such as tools, fingers, clothes, etc. Medical personnel and nail technicians can ensure the appropriate preparation and amount of application of the UV adhesives without rushing to avoid premature curing of the adhesive layer.

The UV adhesives may be applied by an applicator such as a nail brush, spray bottle, or a springe. After the UV adhesives settles on the dispersion layer, UV light is flashed to cure the UV adhesive layer. The UV adhesive layer bonds with the dispersion layer, debrided nail plate, and/or nail bed. Additional layers of the UV adhesive may be applied to achieve desire coverage and thickness. The sealing ability of the UV adhesive layer may reduce the moisture and air around the infected area, thereby reducing a condition that promotes fungal growth. The UV adhesive layer also forms a hard surface, which protects the infected nail from external trauma and cosmetically covers the infected nail.

The UV adhesive layer may also include anti-fungal agents. The anti-fungal agent may or may not be the same anti-fungal agent contained in the dispersion layer. The adhesive layer with anti-fungal agent furthers treats and eliminates the spread of the fungal infection further creating a fungistatic environment.

The UV adhesive layer also provides a hard surface layer for a nail technician to cosmetically treat the infected nail by trimming and shaping the dispersion layer and UV adhesive layer, and then applying a nail polish to the treated nail, if so desired. In lieu of the nail polish, an artificial nail also known as false nails, nail extensions, nail veneers, nail enhancements, etc. may be placed on the adhesive layer.

For further treatment, the patient may be prescribed an oral anti-fungal medication such as terbinafine. The fungistatic environment created by the dispersion layer and UV adhesive layer provides synergistic effect with the oral medication.

An exemplary embodiment of treating a fungal infected nail treatment will now be described. A patient suspected of having a fungal nail infection, for example, onychomycosis, seeks medical assistance from a medical professional. The medical professional determines whether the patient has a fungal nail infection by visual inspection or by performing a diagnostic test such as KOH. In lieu of the visual inspection or diagnostic test, the medical professional may review the medical history of the patient to determine whether the patent had previously been infected with the fungal nail infection.

After confirming that the patient has onychomycosis or similar fungal infection, the medical professional prescribes a treatment regimen. The treatment regimen includes prepping the patient's infected nail for treatment. The infected nail may be soaked in warm water. Dead skins around the infected nail are gently removed. If required, the infected nail may further be trimmed. The infected nail is debrided. The goal of the debridement step is to remove loose debris and as much of the encapsulated fungus as possible. The debridement may remove a portion or the entire nail plate depending on the severity of the infection. The debridement may be mechanical or chemical, e.g., 40% urea. Prior to, as part of or after the debridement step, the infected nail may be scrubbed with baking soda or similar abrasive material. Citric acid may also be used to clean and provide additional anti-fungal treatment. Sodium percarbonate may also be used for cleaning and bleaching the discolored infected nail. The debrided nail is further cleaned with 70% isopropyl alcohol for disinfection.

A dispersion layer is applied to the debrided nail. The dispersion layer may be a TDDS layer or layer of a topical application. The dispersion layer may contain an effective amount of an anti-fungal agent, such as, terbinafine 1% or prescription strength ciclopirox 8%.

After the application of the dispersion layer, a UV adhesive is applied to the dispersion layer. After the UV adhesive settles, UV light is flashed to cure the UV adhesive. Additional layers of the UV adhesive may be applied to get the desire thickness, appearance, and strength. Additional layers of the UV adhesive may be applied if a surface depth of the debridement nail and or the dispersion layer is uneven. An infected nail may only be partially infected, and thus the debridement only removes a portion of the nail, thereby creating an uneven depth on the infected nail. The additional application of the UV adhesive will level the uneven depth and provide full coverage on the surface of the debrided nail. The UV adhesive layer removes air and moisture around the debrided nail. The UV adhesive layer locks the dispersion layer in place, and the hardened UV adhesive layer provides protection from external trauma. The adhesive layer may also contain an additional effective amount of the anti-fungal agent terbinafine 1%, ciclopirox 8% or similar medication.

The UV adhesive layer may be further trimmed, grounded, and polished for cosmetic treatment. An application of nail polish may be applied to the UV adhesive layer if desired.

As fungal infections are difficult to eradicate and have a high recurrence rate, the patient may be further prescribed an oral anti-fungal infection medication such as Terbinafine or a suitable alternative depending on comorbidities, or drug-to-drug interactions. The patient returns as necessary for progress checks and reapplication.

Although embodiments of the present invention have been described herein, it should be understood that the foregoing embodiments and advantages are merely examples and are not to be construed as limiting the present invention or the scope of the claims. Numerous other modifications and embodiments can be devised by those skilled in the art that will fall within the spirit and scope of the principles of this disclosure, and the present teaching can also be readily applied to other types of apparatuses. More particularly, various variations and modifications are possible in the component parts and/or arrangements of the subject combination arrangement within the scope of the disclosure, and the appended claims. In addition to variations and modifications in the component parts and/or arrangements, alternative uses will also be apparent to those skilled in the art.

What is claimed is:

1. A method of treating a fungal nail infection, comprising the steps of:
debriding a nail plate of a fungal infected nail to remove nail plate debris and an encapsulated fungus;
applying a dispersion layer impregnated with a first anti-fungal agent on the debrided nail to inhibit a growth and spread of the fungal infection and to create a fungistatic environment;
applying a UV adhesive layer on the dispersion layer to cover the dispersion layer and the debrided nail to seal the dispersion layer and to add depth and rigidity;
curing the UV adhesive layer with UV light; and
cosmetically treating the fungal infected nail by trimming and shaping the dispersion layer and UV adhesive layer.

2. The method of claim 1, wherein the UV adhesive layer is impregnated with a second anti-fungal agent, and wherein the second anti-fungal agent is the same or different than the first anti-fungal agent.

3. The method of claim 2, wherein the first anti-fungal agent and the second anti-fungal agent is one of terbinafine 1%, tolnaftate 1%, ciclopirox 8%, efinaconazole 10%, and tavaborole 5%.

4. The method of claim 1, wherein a patient seeks medical diagnosis and treatment for the fungal nail infection on an on-line telehealth service website.

5. The method of claim 1, wherein the debridement step further includes:
scrubbing at least one of 40% urea, baking soda, an abrasive material, sodium percarbonate, and citric acid on the fungal infected nail; and
cleaning the fungal infected nail with isopropyl alcohol.

6. The method of claim 1, wherein the dispersion layer is a transdermal drug delivery system (TDDS) layer, and wherein the TDDS layer is at least one of a mesh, a transdermal patch, microsponages, liposomes, and fiberglass.

7. The method of claim 1, wherein the dispersion layer is a layer of an alcohol-based terbinafine 1%.

8. The method of claim 1, wherein supplies for the dispersion layer and the UV adhesive layer are provided in a treatment kit.

9. The method of claim 1, wherein the UV adhesive is an epoxy resin or acrylate.

10. The method of claim 1, further comprising applying a nail polish on the cosmetically treated nail.

11. A method of treating a fungal nail infection, comprising the steps of:
debriding a nail plate of a fungal infected nail to remove nail plate debris and an encapsulated fungus, wherein the debriding step further includes scrubbing at least one of baking soda, an abrasive material, sodium percarbonate, and citric acid on the fungal infected nail;
cleaning the debrided nail with isopropyl alcohol;
applying a mesh dispersion layer impregnated with a first anti-fungal agent on the debrided nail to inhibit the growth and spread of the fungal infection and to create a fungistatic environment;
applying a UV adhesive layer on the dispersion layer to cover the mesh dispersion layer and the debrided nail to seal the dispersion layer and to add depth and rigidity;
curing the UV adhesive layer with UV light; and
cosmetically treating the fungal infected nail by trimming and shaping the mesh dispersion layer and UV adhesive layer.

12. The method of claim 11, wherein the UV adhesive layer is impregnated with a second anti-fungal agent, wherein the second anti-fungal agent is the same or different than the first anti-fungal agent.

13. The method of claim 12, wherein the first anti-fungal agent and the second anti-fungal agent is one of terbinafine 1%, tolnaftate 1%, ciclopirox 8%, efinaconazole 10%, and tavaborole 5%.

14. The method of claim 11, wherein the debridement step is a chemical debridement step using 40% urea.

15. The method of claim 11, further comprising applying a nail polish on the cosmetically treated nail.

16. The method of claim 11, wherein the UV adhesive is an epoxy resin or acrylate.

17. A method of treating a fungal nail infection, comprising the steps of:
debriding a nail plate of a fungal infected nail to remove nail plate debris and an encapsulated fungus, wherein the debriding step further includes scrubbing at least one of baking soda, an abrasive material, sodium percarbonate, and citric acid on the fungal infected nail;
cleaning the fungal infected nail with isopropyl alcohol;
applying an alcohol-based anti-fungal agent topical layer with on the debrided nail to inhibit the growth and spread of the fungal infection to create a fungistatic environment;
applying a UV adhesive layer impregnated with an anti-fungal agent on the alcohol-based anti-fungal agent topical solution layer to cover the alcohol-based anti-fungal agent topical solution layer and the debrided nail to seal and to add depth and rigidity, wherein the anti-fungal agent is the same or different than an anti-fungal agent of the alcohol-based anti-fungal agent topical solution layer;
curing the UV adhesive layer with UV light; and
cosmetically treating the fungal infected nail by trimming and shaping the dispersion layer and UV adhesive layer.

18. The method of claim 17, wherein the anti-fungal agent of the alcohol-based topical layer and the anti-fungal agent of the UV adhesive layer is one of terbinafine 1%, tolnaftate 1%, ciclopirox 8%, efinaconazole 10%, and tavaborole 5%.

19. The method of claim 17, wherein the debridement step is a chemical debridement step using 40% urea.

20. The method of claim 17, wherein the alcohol-based anti-fungal topical solution layer is a layer of an alcohol-based terbinafine 1%.
